(19) United States
(12) Patent Application Publication
(10) Pub. No.: US 2013/0116549 A1
(43) Pub. Date: May 9, 2013

(54) Anchored Working Channel

(71) Applicants: Erhan H. Gunday, Great Neck, NY (US); Lawrence J. Gerrans, San Anselmo, CA (US); Alex Hsia, San Jose, CA (US)

(72) Inventors: Erhan H. Gunday, Great Neck, NY (US); Lawrence J. Gerrans, San Anselmo, CA (US); Alex Hsia, San Jose, CA (US)

(21) Appl. No.: 13/726,340

(22) Filed: Dec. 24, 2012

Related U.S. Application Data
(63) Continuation-in-part of application No. 12/906,736, filed on Oct. 18, 2010.

(51) Int. Cl. A61B 1/32; A61M 37/00; A61B 1/005; A61B 5/06; A61B 1/00; A61B 1/01

(52) U.S. Cl. CPC A61B 1/32; A61B 1/00154; A61B 1/00165; A61B 1/01; A61B 1/0051; A61B 1/0057; A61B 5/064; A61B 1/00082; A61M 37/00
USPC 600/424; 600/114; 600/115; 600/204; 604/96.01; 604/513

(57) ABSTRACT

An anchored working channel includes an elongated shaft with a proximal end and a distal end, and at least one inflatable balloon positioned at the distal end of the elongated shaft and having an outer wall having an outer surface for contacting surrounding tissue, wherein the elongated shaft has a first lumen through which fluid is supplied to inflate the at least one inflatable balloon such that the balloon anchors the shaft to surrounding tissue, wherein the elongated shaft has a second lumen that accommodates at least one medical instrument and/or device inserted therein, and wherein the outer surface of the at least one inflatable balloon comprises a textured surface for preventing slippage of the outer surface on surrounding tissue.

### FIELD OF THE INVENTION

The present invention relates to systems and methods for anchoring a working channel in a patient's body for deployment and/or use of surgical instruments and devices. More specifically, the present invention relates to a working channel with an expansion apparatus for securing the working channel at a desired location and orientation for precise and minimally traumatic insertion and positioning of catheters, surgical instruments, devices and implants in bodily cavities.

### BACKGROUND OF THE INVENTION

In modern medical practice, there is extensive use of various types of catheters, instruments, devices and implants for various medical procedures. Medical science is increasingly adopting minimally invasive technologies to address and remedy various pathologies and disease states affecting the human body. One of the advantages of such minimally invasive technologies is that they can be done through smaller keyhole incisions, stab punctures and/or through natural orifices of the body into cavities and vessels in the body. Such methods are intended to mitigate trauma to the body and to expedite patient recovery.

Various medical instruments, devices and implants that are transported into and out of the body through these minimally invasive incisions are typically small in diameter, linear and, consequently, can be difficult to guide and navigate into, through, and out of the body. There are several methods for introducing such instruments and devices into a patient's body.

One of the methods utilizes a flexible guidewire over which the desired medical or surgical instrument is introduced. The medical community has long used guide wires to address the difficulties of exacting the location and placement of medical instruments, devices and implants. Coring, reaming, cutting and dilation devices, such as drills, reamers, dilators, taps, shears, energy delivery tools and similar instruments, are often guided into a desired position over a guide wire to open or create new passages into the body. Imaging devices such as cameras, scopes, probes and illumination fibers have been known to be placed over guide wires. Implants, such as stents, bone screws, intra-medullar rods, soft tissue anchors, valves and various other implants are commonly placed over guide wires. Commonly, the tubular structures of the body are intervened with devices known as catheters that are placed and delivered over guide wires.

While guidewires are very useful in minimally invasive medical procedures, they present a number of disadvantages. One of the disadvantages is that the guidewires are only utilized during the insertion of various instruments into bodily cavities, but have to be withdrawn once the instrument is inserted and thus, are not useful during performance of surgical procedures. In order to be able to introduce other devices necessary to carry out a procedure, such as irrigation and suction channels, imaging and illumination devices, etc., a catheter or endoscope with a working channel has to be introduced into the patient's body. Further limitations of the guide wires include difficulty of precise positioning of the medical devices in a desired location, as the guide wire will often move away from the target site during the insertion of the devices. Yet another limitation is that many of the guide wires do not provide imaging capabilities, thereby making the insertion and positioning of the guide wire and other devices very difficult for a surgeon.

Another method of introducing various medical devices into a patient's body is through a working channel of a catheter or an endoscope. Most endoscopes and catheters currently include at least one of a plurality of working channels which extend along the length of the endoscope or catheter to provide access to body tissue within the body cavity. These working channels typically include a rigid non-bendable section and a flexible bendable section. The working channels allow for air insufflation, water flow, suction, and introduction of other medical devices.

Although conventional catheters and endoscopes utilize a wide variety of materials for the working channels, all of them typically require the working channel to be an integral part of the device. Because catheters and endoscopes are subjected to repeated use and are required to follow tortuous pathways within the body, a frequent cause of failure of the working channel is the bending, kinking or fracture of a section of the working channel. This renders the catheter or endoscope useless until it is repaired, which requires disassembly of the device and replacement of the working channel.

Another limitation in the utility of the catheters and endoscopes is that their outer diameters are often too large, thereby making them inadequate for use in the far reaches of the body's organs, vessels and spaces, and furthermore, their inner working channels' diameters are often too small. Optimizing the external and internal diameters of the catheter or endoscope is limited by the size and requirements of the mechanical structures required for the articulation and/or operation of the catheter/endoscope, such as wires, optics, channels, etc.

Yet another disadvantage of known catheter and endoscope devices with working channels is that, once the catheter/endoscope is introduced in a patient's body and a surgical procedure is commenced, the catheter/endoscope will often migrate away from the surgical site, thereby making it difficult for a surgeon to carry out the procedure and requiring further repositioning of the device.

There have been some attempts to overcome the problems of known working channel devices. For example, U.S. Pat. No. 5,938,585 to Donofrio describes an endoscope with an anchoring and positioning device, in the form of an inflatable balloon, at its distal end. The endoscope includes an illumination source and an imaging device at its distal end. The inflatable balloon includes a window portion therein for accommodating an imaging device and is shaped such that it provides space between the imaging device and the cavity wall when inflated so that the cavity wall may be viewed by the imaging device.

U.S. Patent Publication No. 2011/0004058 to Oneda et al. describes an imaging endoscope having an outer shaft and an inner shaft movable therein. The endoscope further includes an imaging capsule mounted on a distal end of the inner shaft. The outer shaft or the imaging capsule may include an inflatable balloon at the distal end to anchor the imaging unit in a bodily cavity.

U.S. Patent Publication No. 2004/0230219 to Roucher Jr. describes an anchoring, supporting and centering catheter system for treatment of coronary artery disease. The system includes a balloon sheath apparatus having an inflatable balloon at its distal end, a guidewire lumen and an inflation lumen. The balloon sheath is used to facilitate the centering of the guidewire into an occlusion in the blood vessel. The system also includes a hydraulic guidewire that is inserted through the guidewire lumen of the balloon sheath, and an exchange sheath that is extended over the guidewire to further dilate the occlusion.

U.S. Pat. No. 5,484,412 to Pierpont describes an angioplasty catheter including a balloon dilatation catheter positioned inside an anchoring catheter, which in turn is positioned inside a guiding catheter. The guiding catheter is inserted into an artery, then the anchoring catheter is extended out of the guiding catheter and anchored to the artery wall by inflating the external balloons, and then the dilatation catheter is extended out of the anchoring catheter to perform an angioplasty procedure.

U.S. Patent Publication No. 2009/0076447 to Casas et al. described a flexible catheter with an inflatable balloon at its distal end, the catheter including a wire lumen and a balloon inflation lumen. The flexible catheter with a guide wire is inserted to a target site, the guide wire is advanced through the catheter to an anchor location, and the flexible catheter is withdrawn, leaving the guide wire in place. Then, an anchor catheter is inserted over the guide wire, the guide wire is withdrawn, and the balloon is inflated to anchor the catheter at the site. Another guide wire can then be inserted through the anchor catheter, the balloon is deflated, and the anchor catheter is withdrawn from the bodily cavity.

While these known devices provide some improvements over the older systems, they still suffer from significant disadvantages. One of the major problems with the prior art systems described above is that is that they are rather bulky and complex in structure, which makes them unsuitable for use in bodily cavities having a very small diameter, such as lungs. Additionally, these known systems are typically constructed with expensive materials and require multiple working components, and therefore have to be reused multiple times, which requires complex sterilization procedures.

Another problem is that the devices described above often migrate from the desired location during the insertion and operation of the devices. This is because the only securing mechanism holding the devices in place is the contact between the inflated balloon and surrounding cavity walls. The prior art devices have balloons with a smooth surface, thereby making them prone to slippage during the operation of the devices due to linear and/or rotational forces exerted upon the devices.

A further deficiency of the prior art working channel devices is that they are not capable of being positioned as optimally and precisely as may be desired. The known devices do not provide a direct visual feedback of the area ahead, behind, and around the working channel to optimize positioning and operation of the device.

Yet another shortcoming of the known working channel devices is that they lack the capability to precisely gauge the size of the environment in which they are being used to provide physiological measurements and feedback that could aid precise and secure positioning and operation of the device. For example, the prior art devices do not enable the surgeon to measure the intra-lumen diameter of the bodily cavity in which the working channel is to be secured operated, and provide no way to accurately adjust for changes in this diameter during the procedure. Because the known devices have no mechanism for measuring the intra-lumen diameter at different points within the cavity, the surgeon is not able to properly adjust the amount of pressure supplied to the anchoring balloon and thereby prevent slippage or migration of the balloon.

What is desired, therefore, is an improved anchored working channel that addresses the disadvantages and shortcoming of the prior art systems described above.

### SUMMARY OF THE INVENTION

It is, therefore, an object of the invention to provide a new and improved anchored working channel that overcomes the problems of known devices.

It is also an object of the invention to provide a new and improved anchored working channel that addresses the dislocation, migration and instability problems of the prior art devices.

It is another object of the invention to provide a new and improved anchored working channel that provides improved imaging capabilities to enable more precise positioning and operation of the device.

It is further an object of the invention to provide a new and improved anchored working channel that may be used with existing catheter and endoscope devices.

It is yet another object of the invention to provide a new and improved anchored working channel that is simple in structure and is capable of being used in bodily cavities having smaller diameters.

In order to achieve at least the above-mentioned objects of the present invention, an anchored working channel is provided, comprising an elongated shaft with a proximal end and a distal end, and at least one inflatable balloon positioned at the distal end of the elongated shaft and having an outer wall, said outer wall comprising an outer surface for contacting surrounding tissue, wherein the elongated shaft has a first lumen through which fluid is supplied to inflate said at least one inflatable balloon such that said at least one balloon anchors the shaft to surrounding tissue, wherein the elongated shaft has a second lumen that accommodates at least one medical instrument and/or device inserted therein, and wherein said outer surface of said at least one inflatable balloon comprises a textured surface for preventing slippage of the outer surface on surrounding tissue.

In certain embodiments, the textured surface of the at least one inflatable balloon comprises a mesh disposed on the outer wall of the balloon. In some of these embodiments, the mesh is a weft knit mesh. In additional of these embodiments, the mesh comprises polyethylene. In further of these embodiments, the mesh comprises elastane.

In some embodiments, the anchored working channel further includes an imaging device disposed in one of the first lumen and the second lumen. In certain of these embodiments, a distal end of said imaging device extends out from the distal end of said elongated shaft for viewing tissue in front of the anchored working channel. In further of these embodiments, the imaging device comprises a fiber optic bundle.

In certain embodiments, the imaging device comprises a steerable distal section. In some of these embodiments, the imaging device further includes a control unit for actuation of the steerable distal section by a user. In further of these embodiments, the imaging device comprises an inner lumen and a plurality of steering lumens. In certain of these embodiments, the imaging device further comprises at least one pull wire disposed in at least one of the plurality of steering lumens for actuation of the distal section of said imaging device.

In certain embodiments, the fluid is a gas.

In some advantageous embodiments, the fluid is supplied to the at least one balloon by a pump. In certain of these embodiments, the pump is an electro-pneumatic pump. In additional of these embodiments, the pump further comprises a vacuum source that evacuates the fluid from said at least one inflatable balloon. In further of these embodiments, the pump includes at least one sensor for measuring at least one parameter and a processor that controls the supply of the fluid to said at least one inflatable balloon based on the at least one measured parameter. In yet further of these embodiments, a data device is provided from which the pump identifies a particular type of the working channel connected thereto.

In certain embodiments, the at least one inflatable balloon comprises at least one imaging marker. In some of these embodiments, the at least one imaging marker comprises a radio-opaque ring.

In some cases, the proximal end of said elongated shaft comprises a first port in communication with the first lumen and at least one second port in communication with the second lumen.

In certain embodiments, the elongated shaft further comprises a bypass lumen in fluid communication with an opening in the elongated shaft positioned proximally from said inflatable balloon for passing bodily fluids therethrough.

In certain advantageous embodiments, the at least one inflatable balloon comprises a plurality of inflatable balloons positioned at different locations along said elongated shaft. In some of these embodiments, each of the plurality of inflatable balloons is inflatable separately from the other balloons.

In some embodiments, the medical instrument and/or device is a resecting balloon catheter. In other embodiments, the medical instrument and/or device is a steerable catheter. In yet further embodiments, the medical instrument and/or device is a fiberscope.

In certain embodiments, the working channel further includes at least one opening in the outer wall of the elongated shaft for delivering a therapeutic and/or diagnostic agent to surrounding tissue.

A method of performing a medical procedure via an anchored working channel is also provided, including the steps of inserting a working channel into a bodily cavity, wherein said working channel comprises an elongated shaft having at least a first lumen and a second lumen therein, and an inflatable balloon positioned at a distal end of the elongated shaft and having an outer wall with a textured surface for preventing slippage of the outer wall on surrounding tissue, advancing said working channel through the bodily cavity until the inflatable balloon reaches an anchoring position, supplying fluid to the first lumen with a pump until the balloon is inflated such that the textured surface exerts sufficient pressure on the wall of the bodily cavity to retain the balloon in the anchoring position, inserting at least one medical instrument and/or device through the second lumen and out of the distal end of said elongated shaft for performing the medical procedure, withdrawing the at least one medical instrument and/or device from the second lumen, deflating the inflatable balloon, and withdrawing the working channel from the bodily cavity.

In some embodiments, the pump includes at least one sensor for measuring at least one parameter and a processor for controlling the supply of fluid to the inflatable balloon based on at the least one measured parameter.

In certain embodiments, the method further includes the step of using an imaging device disposed in one of the first lumen and the second lumen to visualize tissue in the bodily cavity.

In some cases, the step of using the imaging device comprises extending a distal tip of said imaging device out of the distal end of said elongated shaft to visualize tissue in front of said anchored working channel. In certain of these cases, the imaging device comprises a steerable distal section and the step of using the imaging device comprises actuating said distal section via a control unit to maneuver said imaging device in the bodily cavity.

In certain embodiments, the method further includes the step of using at least one imaging marker to position the inflatable balloon within the bodily cavity.

In some embodiments, the elongated shaft comprises a bypass lumen in fluid communication with an opening in the elongated shaft positioned proximally from the inflatable balloon, and the method further includes the step of passing bodily fluids through the bypass lumen and out of the opening in the elongated shaft. In certain of these embodiments, the method further includes the step of measuring airflow through the bypass lumen.

In certain embodiments, the textured surface of the inflatable balloon comprises a mesh disposed on the outer wall of the balloon. In certain of these embodiments, the mesh is a weft knit mesh. In additional of these embodiments, the mesh comprises elastane.

In some embodiments, the step of advancing the working channel through the bodily cavity comprises the steps of inserting a guide wire into the bodily cavity and advancing the working channel over the guide wire until it reaches the anchoring position.

In certain embodiments, the method further includes the step of delivering a therapeutic and/or diagnostic agent to tissue via at least one opening in the outer wall of the elongated shaft. In some of these embodiments, the step of delivering the therapeutic and/or diagnostic agent to tissue includes at least partially deflating the inflatable balloon and moving the elongated shaft in a proximal direction to facilitate extravasation of the agent into tissue.

These and other objects, advantages and features of this invention will become apparent upon review of the following specification in conjunction with the drawings.

### BRIEF DESCRIPTION OF THE DRAWINGS

FIG. 1A is schematic view of an anchored working channel in accordance with the invention.

FIG. 1B is a schematic view of the anchored working channel of FIG. 1A with a plurality of balloons.

FIG. 1C is a schematic view of the anchored working channel of FIG. 1A, showing various medical instruments inserted therethrough.

FIG. 1D is a schematic view of the anchored working channel of FIG. 1A, showing a proximal end of the working channel in more detail.

FIG. 2 is an end view of the inflated balloon of the anchored working channel of FIG. 1A.

FIG. 3A is a perspective cross-sectional view of a distal end of the anchored working channel of FIG. 1A.

FIG. 3B is a plan cross-sectional view of a distal end of the anchored working channel of FIG. 1A.

FIG. 4 is a partially schematic view of the working channel of FIG. 1A, showing connection to a pump.

FIG. 5 is a perspective view of a distal end of the anchored working channel of FIG. 1A, showing an imaging device disposed therein.

FIG. 6 is a cross-sectional view of a distal end of the imaging device of FIG. 5.

FIGS. 7-9 are views of the anchored working channel of FIG. 1A being operated in a bodily cavity.

### DETAILED DESCRIPTION OF THE INVENTION

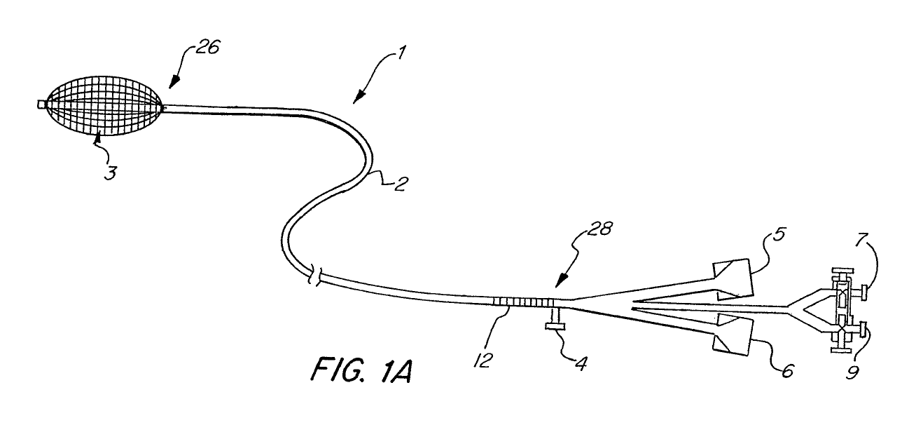

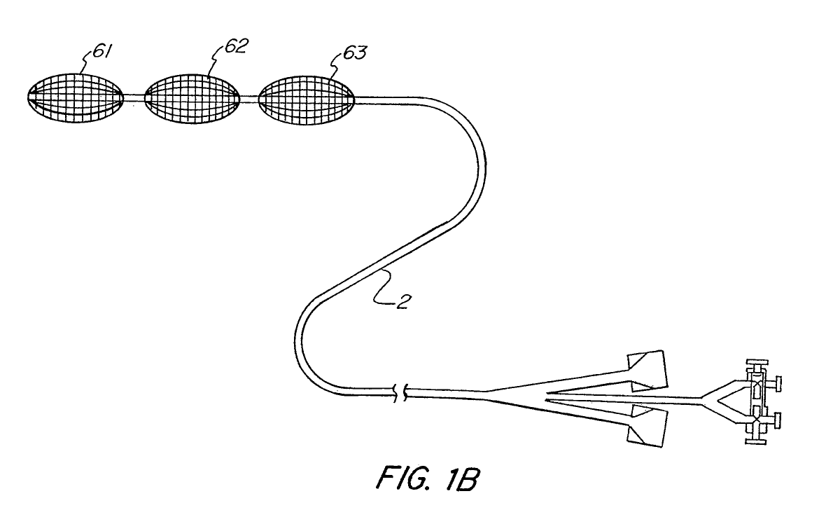

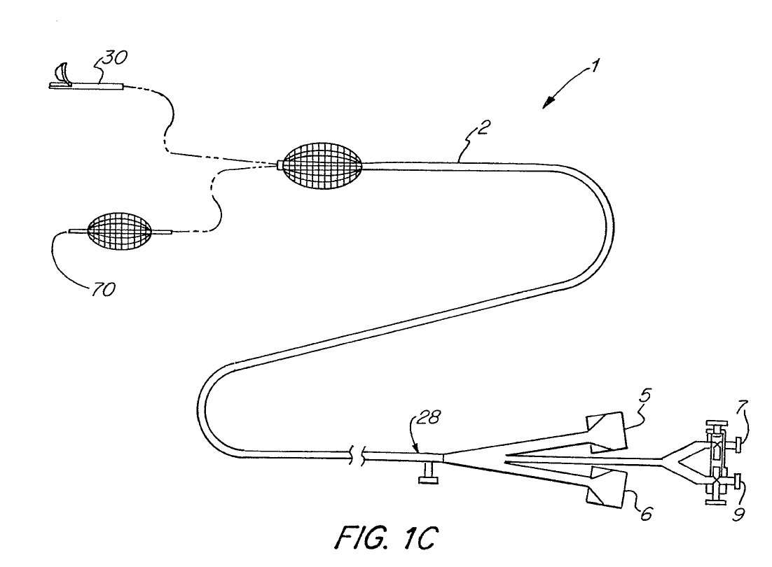

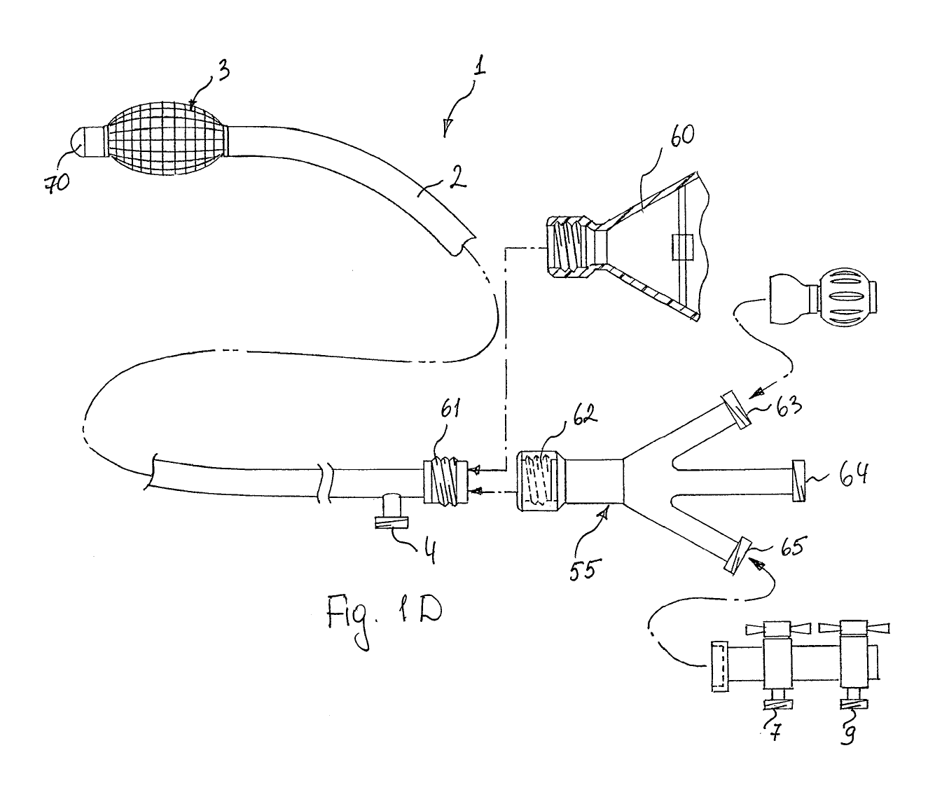

The basic components of one embodiment of an anchored working channel in accordance with the invention are illustrated in FIG. 1A. As used in the description, the terms “top,” “bottom,” “above,” “below,” “over,” “under,” “above,” “beneath,” “on top,” “underneath,” “up,” “down,” “upper,” “lower,” “front,” “rear,” “back,” “forward” and “backward” refer to the objects referenced when in the orientation illustrated in the drawings, which orientation is not necessary for achieving the objects of the invention.

The anchored working channel of the present invention may be used with various catheter or endoscope devices, various types of surgical instruments, tools, and operative devices, implants and related medical diagnostic and treatment systems that need to be inserted into bodily cavities and operated therein via a suitable working channel. In an advantageous embodiment, the anchored working channel is used with a resector balloon system described in U.S. Pat. No. 8,226,601, the disclosure of which is incorporated by reference herein in its entirety. In another advantageous embodiment, the working channel of the present invention is used with a steerable catheter system described in U.S. patent application Ser. No. 13/037,874, the disclosure of which is also incorporated by reference herein in its entirety. In yet another advantageous embodiment, the working chanel is used with an anchored guidewire described in U.S. patent application Ser. No. 12/906,736 the disclosure of which is also incorporated by reference herein in its entirety.

As shown in FIG. 1A, the anchored working channel (1) includes an elongated shaft (2) having a distal end (26) and a proximal end (28). The shaft (2), which can be rigid or flexible, may have any suitable diameter and length depending on a particular application and/or dimensions of target bodily cavity, and may be flexible, rigid or semi rigid. In one advantageous embodiment of the present invention, the elongated shaft has a length of about 90 mm, an inner diameter of about 4 mm and an outer diameter of about 4.5 mm.

The elongated shaft (2) may be made with any commercially available material that is flexible enough to allow the shaft to be safely inserted through the available opening of a bodily cavity such that it will deflect from the walls of the cavity instead of puncturing them. In particular, a distal end section of the elongated shaft (2) is made flexible to ensure safe insertion of the working channel into bodily cavities.

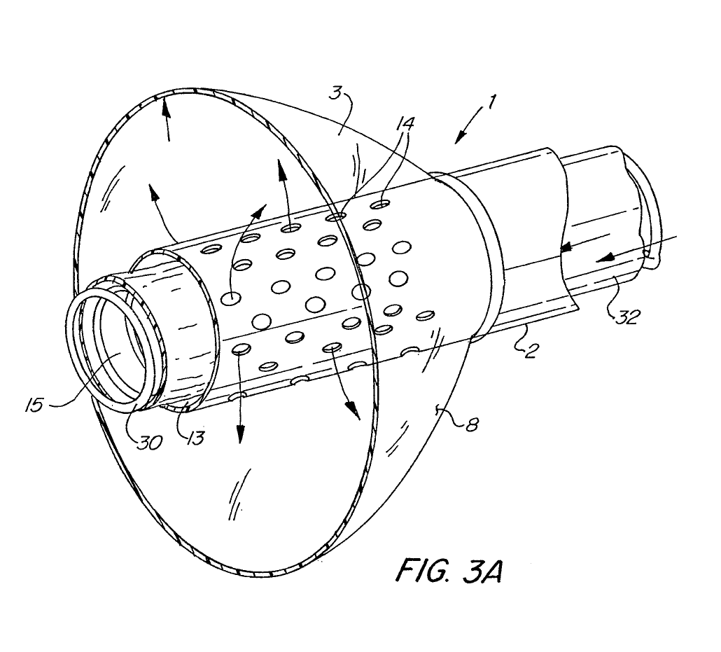

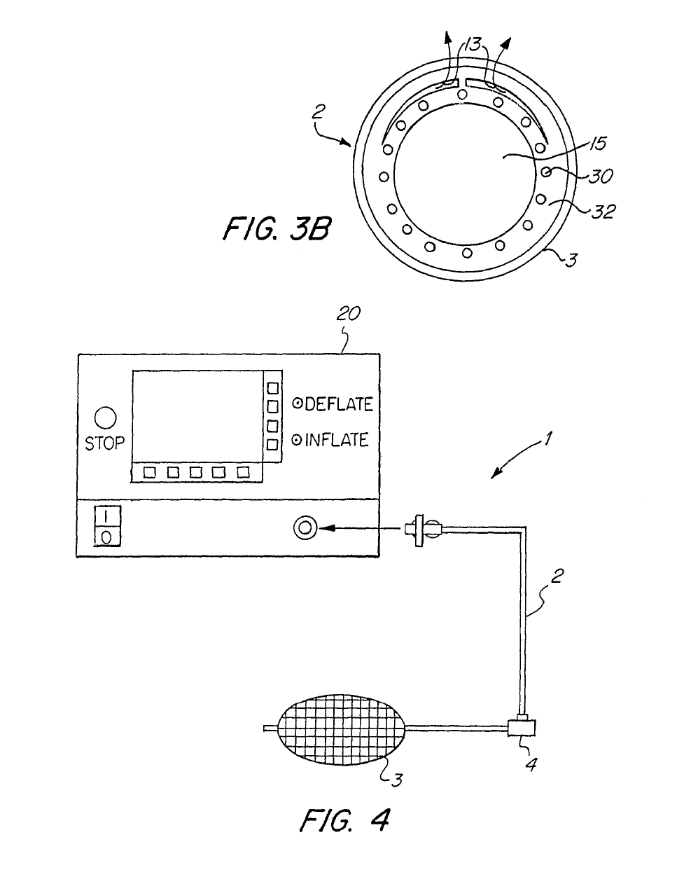

In some embodiments, the shaft (2) may include a coating made of suitably smooth material to facilitate the movement of the working channel through the bodily cavities. In one advantageous embodiment shown in FIG. 3, the elongated shaft (2) consists of a coil wire (30) made of any suitable material, such as stainless steel, and a coating (32) made of suitable materials, such as polyethylene, polyurethane, Pebax® and the like. A braided sheath may also be used instead of the coil wire. In some advantageous embodiments, the coil wire or the braided sheath may be made with a memory shape material, such as nitinol.

In further advantageous embodiments, the elongated shaft may include a combination of braided sheath and coil wire materials to provide for optimal flexibility and maneuverability of the shaft. For example, a distal portion of the elongated shaft may be made with coiled wire material and thus, have more flexibility, and the rest of the elongated shaft is made with the braided sheath material and less flexible.

The coil wire (30) or braid can be molded over during the shaft extrusion process and can run the entire length of the elongated shaft (2). Alternatively, the elongated shaft (2) may be molded or extruded in a first step and the coil wire (30) may be disposed within an inner lumen of the shaft. Such design improves torque, maneuverability, and kick resistance of the elongated shaft (2), and also prevents reduction of the working channel diameter.

The elongated shaft (2) may, as shown in FIG. 1A, further include calibrated markings (12) to gauge extent of insertion of the shaft (2) into a bodily cavity. In some embodiments, the elongated shaft (2) may further include imaging markers positioned at the distal end (26) of the shaft or at any other location along the shaft to facilitate external imaging thereof and thereby allow for better visualization during insertion and positioning of the working channel (1) in bodily cavities.

The distal end of the elongated shaft (2) includes at least one inflatable balloon (3) located at or near the tip of the distal end. The inflatable balloon (3) has an outer wall with a textured surface, which, when inflated, grips the surrounding tissue in a bodily cavity. The inflatable balloon (3) may be made of latex, Yulex, polyethylene, nylon or other suitable material, and may come in a variety of sizes and diameters, which allow the working channel (1) to be used in bodily cavities of various diameters and dimensions, such as large and small bronchial branches, sinuses, vessels, etc. In some advantageous embodiments, the inflatable balloon (3) has a length of about 10 mm and a diameter of about 10 mm. In certain embodiments, a compliant balloon is employed. In further advantageous embodiments, the inflatable balloon (3) may comprise a plurality of balloons/bladders, which may be controlled, inflated and deflated independently of each other.

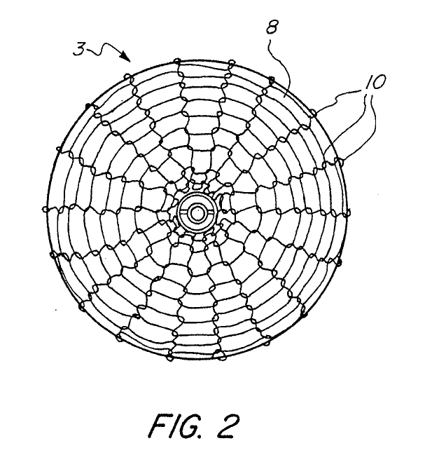

FIG. 2 illustrates an end view of the inflated balloon (3) of the anchored working channel (1). The outer surface (8) of the balloon (3) includes a woven mesh (10) disposed on the outer surface of the balloon. The mesh may be made of elastane, latex, polyurethane, composite springs, metallic fibers, elastic, steel fibers, or other appropriate material, or a composite or coating thereof. In some advantageous embodiments, the mesh is made with elastane material. In particularly advantageous embodiments, the mesh is weft knit. In is understood, however, that the mesh sleeve may be made using any suitable mesh manufacturing techniques.

The woven mesh sleeve (10) may be disposed on the outer surface of the balloon (3) by using any suitable manufacturing method. Alternatively, woven sleeve (10) may be knitted or woven from thread directly onto the balloon (3). In some advantageous embodiments, the woven mesh (10) may be affixed to the surface of the balloon (3) during the molding process, which produces outwardly-facing protrusions on the outer surface of the balloon (3) that assist in gripping of the balloon to the surrounding tissue. In other advantageous embodiments, dimensional surface structures, such as bumps or inflatable sinuses, that are encapsulated in the surface substrate of the balloon (3) may be used to produce the surface protrusions forming the textured surface.

The protrusions forming the textured surface of the balloon (3) can have various shapes and configurations, depending on a particular application. In some embodiments, the outer surface of the balloon (3) may have outwardly extending protrusions forming a lattice-like structure or a spiral-like pattern extending circumferentially on the outer surface of the balloon (3). In other embodiments, the protrusions may be in a form of dimples that extend outwardly from the outer surface of the balloon (3). It should be noted that any other shapes and configurations of the surface protrusions can be used in accordance with the present invention, including combinations of any of the aforementioned or other textures.

In certain advantageous embodiments, the balloon (3) includes imaging markers, such as radio opaque rings, located at or near the ends thereof. Such markers can be selected and appropriately positioned in order to reflect or block the relevant waves of various imaging modalities (e.g., x-ray) in order to allow the use of such modalities to assist with the precise positioning of the balloon (3) within a bodily cavity. Similarly, the balloon or balloon mesh may include a radiopaque material, such as a mesh made of yarn having radiopaque iron fibers.

In some embodiments, the distal end of the elongated shaft (2) includes a safety tip (70), such as shown in FIG. 1D. The safety tip has a smooth convex shape designed to deflect from bodily tissues and cavity walls during the insertion of the working channel (1) into a patient's body to prevent injuries to the bodily tissues during the insertion. The safety tip may be made with the same materials as the elongated shaft and has an opening therethrough for introduction of various instruments/devices through the working channel (1).

When in use, the working channel (1) is first introduced into a bodily cavity and positioned adjacent the target tissue site. Then, the balloon (3) is inflated such that the woven mesh sleeve (10) covers at least a portion of the balloon outer surface in an expanded state and adds texture, friction, and surface area to the outer surface of the balloon. The crossover points of the fiber threads forming the mesh produce outwardly-facing, small knots or dimples, which grip the surrounding tissue, thereby anchoring the working channel (1) at the target site.

It is understood that the working channel (1) may also include a plurality of anchoring devices positioned at different locations along the elongated shaft (2). The plurality of anchoring devices allow for more precise and secure anchoring of the working channel (1) within the bodily cavity. As shown in FIG. 1B, multiple balloons (61, 62, 63), each with textured surface, such as a mesh, may be positioned along the distal portion of the shaft (2).

In addition to serving as an anchoring device to secure the working channel within the bodily cavity, the inflatable balloon (3) or a plurality of inflatable balloons can also be used to block or prevent fluids from flowing around the balloon in the target bodily lumen, vessel, airway or space.

It should be noted that in certain applications, such as when the working channel device is used in very small bodily cavities or passages, it may not be necessary to utilize an inflatable balloon to anchor the working channel. For example, when the working channel (1) is used in small lung airway passages, the outer diameter of the elongated shaft itself may be sufficient to fixate the working channel inside the passage.

As shown in FIG. 3A, the elongated shaft (2) of the working channel (1) includes at least two inner lumens. An inflation lumen (13) is connected to the fluid source provided at the proximal end of the elongated shaft (2) and is in fluid communication with the interior of the inflatable balloon (3) via a plurality of openings (14) in the shaft wall positioned inside the balloon (3). The fluid source supplies fluid to the inflation lumen (13) and via the openings (14) to inflate the balloon (3).

The elongated shaft (2) further includes a working channel lumen (15). In the embodiment shown in FIG. 3A, the working channel lumen (15) is an inner lumen is positioned inside the outer inflation lumen (13). However, it is understood that any other configuration of the lumens may be used in accordance with the present invention. For example, as shown in FIG. 3B, the elongated shaft (2) may consist of a coating material (32), such as polyethylene or polyurethane, with an embedded coil or braid (30). The elongated shaft (2) includes an inner working channel lumen (15) and one or more inflation lumens (13) provided in the coating material (32) adjacent the working channel lumen (15).

In additional embodiments, the elongated shaft (2) may also be divided into equal or unequal sections representing the inflation lumen and the working channel lumen. Furthermore, it is understood than the elongated shaft (2) may include more than two inner lumens for performing different functions.

The working channel lumen (15) may be used to deploy various medical instruments or devices into the desired part of the airway, vessel, lumen, pleural cavity or other bodily cavity. The working channel lumen (15) may further be divided into a plurality of lumens (not shown), through which an imaging device, an instrument, a device, or a fluid may be placed. The working channel lumen(s) can be used to deliver any number of things to assist a surgeon with performing a surgical or diagnostic medical procedure, such as cutting or resecting tissue, aspiration, respiration, imaging, delivering various therapeutic and/or diagnostic agents, delivering stents, scaffolds or implants, and such.

Referring back to FIG. 1A, the proximal end (28) of the elongated shaft (2) includes an inflation port (4) for connection of the working channel (1) to a fluid source, such as a pump, through which the balloon (3) is inflated. The inflation port (4) is provided with any suitable connector, such as a luer connector, for connection to the pump. The inflation port (4) is in fluid communication with the inflatable balloon (3) via the inflation lumen (13) of the elongated shaft (2).

As shown in FIG. 1C, the proximal end of the elongated shaft (2) further includes one or more ports through which various medical instruments or devices are inserted into the working channel lumen. For example, the proximal end (28) of the elongated shaft (2) includes an imaging device port (5), an instrument port (6), a suction port (7) and an irrigation port (9). The imaging device port (5) is used for insertion of an imaging device (30), as discussed in more detail below. The instrument port (6) provides an access point for insertion of catheters, endoscopes, various surgical or diagnostic medical devices, and the like. The camera port (5) and the instrument port (6) may connect to the same working channel lumen or may be each connected to a separate inner lumen provided in the elongated shaft (2).

In the embodiment illustrated in FIG. 1C, a resecting balloon catheter system described in U.S. Pat. No. 8,266,601 is inserted through the instrument port (6) to perform a desired procedure within the bodily cavity. Preferably, a length of the catheter is sufficiently greater than the length of the elongated shaft (2) and the outer diameter of the catheter is sufficiently smaller than the inner diameter of the working channel lumen such that the catheter may be easily inserted into the lumen and extended out of the distal end (26) of the shaft. In some advantageous embodiments, the length of the catheter operated through the working channel (1) is about 120 mm. It is noted that any other catheter system, such as a balloon catheter, a drug delivery catheter, a steerable catheter, etc., may be used with the working channel of the present invention.

The suction and irrigation ports (7 and 9) function to deliver/suction irrigation fluid to the surgical site. The ports (7, 9) are provided with trumpet valves or any other suitable valve type and are connected to an irrigation fluid/vacuum source positioned outside of the patient's body. The irrigation fluid may be accommodated in the working channel lumen(s) (15), or, alternatively, may be provided via a separate lumen of the elongated shaft (2). In some advantageous embodiments, the suction/irrigation valves are provided in an in-line arrangement to facilitate passage of debris out of the working channel (1).

The proximal section (55) of the elongated shaft (2) may be provided as a separate structure removably attachable to the proximal end of the elongated shaft, as shown in FIG. 1D. This way, the same proximal end section (55) may be used with various working channel devices, and may be easily removed for sterilization or replaced with another attachment (60), e.g. bronch adapter shown in this figure, desired for a specific medical procedure. In the exemplary embodiment shown in FIG. 1D, a threaded connector is used to attach the proximal section (55) to the proximal end of the elongated shaft. An external thread (61) is provided on the outer surface of the elongated shaft and a corresponding internal thread (62) is provided on the inner surface of the proximal section (55). It is understood, however, that any other suitable connection mechanism may be used to connect the proximal section (55) to the elongated shaft (2).

The proximal section (55) includes various ports, e.g. an imaging device port (63), an instrument port (64), a suction/irrigation port (65), etc., for connection to or insertion of various instruments and/or devices needed to perform a particular procedure. The ports may be provided with any suitable connectors and/or adapters, such as seal lip connector, luer connector, Tuohy Borst type adapter, and the like.

In additional advantageous embodiments, the elongated shaft (2) may further include a bypass lumen to allow bodily fluids, such as air or blood, to flow through the working channel (1), which is necessary in certain medical applications, e.g. pulmonology or cardiology. In the case of air bypass, the air may flow through one of the shaft lumens and in/out of the proximal end of the working channel (1) positioned outside of the patient's body. In some cases, an external device, such as a respiration device, is in communication with the shaft lumen in order to help facilitate this flow. If a blood bypass is desired, an additional port/opening may be provided in the elongated shaft (2) towards the distal end of the shaft to allow for blood to flow through one of the shaft lumens and out of the opening. It is understood that a separate bypass lumen is not required and that the working channel lumen(s) (15) may function as a bypass lumen.

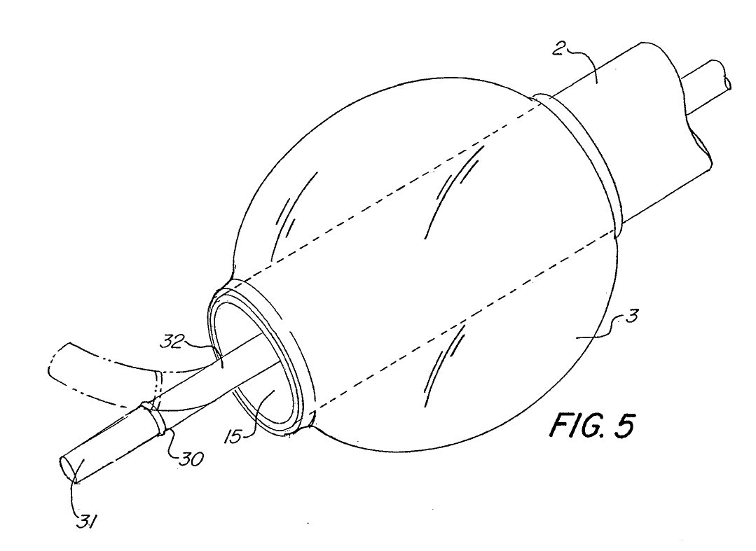

The anchored working channel (1) with the fluid source (20) is further shown in FIG. 4. Any suitable fluid source may be used in accordance with the present invention. In one advantageous embodiment, the fluid source (20) is an electro-pneumatic pump having controls on the front thereof, from which a physician or assistant can control the system (as well as a remote control unit), such as that disclosed in U.S. Pat. No. 8,266,601 to Gunday et al. The pump (20) supplies a fluid, such as a gas, liquid, or mixture thereof, to the inflation lumen (13) of the working channel via the inflation port (4). The pump (20) also includes a variety of capabilities for balloon identification, proper inflation/deflation of the balloon, and feedback measurements, many details of which are described in Gunday et al. In certain advantageous embodiments, the pump (20) further includes a vacuum source to evacuate fluid from the balloon (3). In other embodiments, a handheld pump is used as a fluid source.

In some embodiments, the working channel (1) includes a data device, such as optical, RFID, flash memory, etc. This way, the pump (20) is able to identify the type of working channel device that is connected and read the characterization data of the balloon, e.g. maxim pressure, volume, dimensions, etc., and/or working channel included thereon, and then adjust its control accordingly based on user input.

The pump (20) further includes a processor that controls the supply of fluid to the inflatable balloon (3) based on at least one predetermined parameter. In some embodiments, such predetermined parameters may be manually entered by the user. Alternatively, the control of the fluid is based on default parameters selected by the pump (20), which are based on the characteristics of the particular balloon and/or the diameter measurements of a particular bodily cavity made by the pump. Furthermore, the pump may control and regulate the pressure by monitoring and taking into account one or more vital signs and physiological parameters of the patient, such as body temperature, heart rate, blood pressure, and respiratory rate.

In some advantageous embodiments, the working channel (1) of the present invention is capable of measuring airflow through the bypass lumen of the elongated shaft (2). The airflow may be measured by the pump (20) or by a separate sensor coupled to the bypass lumen of the working channel. This is particularly advantageous in pulmonary applications, where it is important to measure the amount of airflow to and from a patient's lungs.

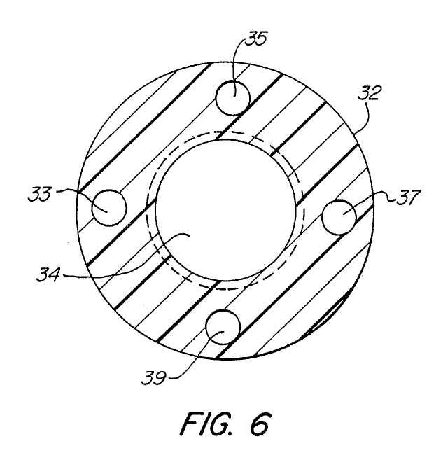

Referring to FIG. 5, the working channel (1) is further provided with an imaging device (30) disposed in the elongated shaft (2). The imaging device is used to facilitate the insertion and positioning of the working channel in the bodily cavity, and may further assist the surgeon in performing a medical procedure. The imaging device (30) is inserted into the working channel lumen (15) through the imaging device port (5) and is extended out of the distal end of the elongated shaft (2) such that the tissue in front of the working channel can be viewed by the imaging device during the insertion of the working channel (1) into a bodily cavity.

The imaging device (30) includes a camera head (31) disposed at a distal end of a sheath (32). The sheath has a length that is sufficiently greater than the length of the elongated shaft (2), such that the imaging device (30) can be extended out of the distal end of the elongated shaft. In some advantageous embodiments, the length of the imaging device sheath (32) is about 105 mm. Additionally, an outer diameter of the imaging device sheath is smaller than the inner diameter of the working channel lumen (15) to facilitate the insertion of the imaging device through the lumen. In one advantageous embodiment, the outer diameter of the sheath (32) is less than about 1 mm. The sheath (32) is preferably made with a flexible material that allows for rotational or linear movement of the distal end of the sheath.

It is understood that the imaging device (30) may also be introduced into a bodily cavity through the inflation lumen (13) of the working channel. This way, the inflation lumen (13) serves a dual purpose—it is used both for supply of fluid to inflate/deflate the balloon (3) and for visualization via the imaging device (30). In these embodiments, an imaging device aperture may be positioned inside the balloon (3), and the outer wall of the balloon is made transparent when inflated, such that imaging is made possible from inside the balloon (3). The imaging device aperture can also serve as an inflation/deflation opening through which the fluid is supplied to/from the balloon (3). Additionally, the elongated shaft (2) may have one or more imaging device apertures positioned at different locations along the shaft for better visualization of the surrounding area during the introduction of the working channel (1) into the patient's body.

In one advantageous embodiment shown in FIG. 5, the imaging device (30) includes a steerable flexible distal tip that can be translated linearly or rotationally inside the bodily cavity. This allows for enhanced visualization of the surrounding area during the insertion and operation of the working channel (1). As shown in FIG. 6, the imaging device sheath (32) includes four steering lumens (33, 35, 37, 39) extending through the entire length of the sheath. It is understood that a lesser or great number of steering lumens may also be provided, depending on the desired level of maneuverability of the imaging device (30). A center lumen (34) is also provided in the sheath (32) for accommodating components of the imaging device (30). The steering lumens (33, 35, 37, 39) are shown integrally formed as part of the sheath (32) and are radially offset from the longitudinal axis of the sheath (32) and the center lumen (34). However, it is understood that any other suitable configuration and/or construction of the sheath and the steerable lumens may be used in accordance with the invention.

In some advantageous embodiments, the distal end of the imaging device (30) is actuated by engaging pull wire(s) disposed in each of the steering lumens (33, 35, 37, 39). In other advantageous embodiments, any one or more of the steering lumens (33, 35, 37, 39) may be filled with pressured air in various amounts. In yet further embodiments, the opposite steering lumen(s) (33, 37) or (35, 39) may be deflated with vacuum to facilitate the movement of the distal tip of the imaging device (30).

There is a control unit positioned outside of a patient's body and connected to the imaging device (30) via the imaging device port (5) to allow for manipulation of the imaging device by a surgeon. The imaging device (30) is further coupled to any suitable type of a processor and a display device for processing the imaging data received from the imaging device and displaying the data to the surgeon. It is noted that the imaging device (30) may also be wirelessly connected to the control unit, the processor and/or the display device.

The distal end of the imaging device sheath (32) has a camera head (31) disposed thereon. In an advantageous embodiment, the imaging device (30) is a fiber optic image bundle. Two separate fiber optic bundles—an incoherent fiber bundle for illumination and a coherent fiber bundle for imaging—can also be used in accordance with the present invention. It should be noted that a suitable image sensor (e.g. CCD or CMOS) can be positioned at the tip of the imaging device (30), eliminating the need for a coherent imaging fiber bundle, thus increasing the image quality and reducing cost. It should also be noted that other sources of illumination, such as light emitting diodes, can be employed.

In some embodiments, a fiberscope device may be used in addition to the imaging device (30) for providing enhanced visualization of the target site. The fiberscope is inserted into the working channel lumen (15) of the elongated shaft (2) through the instrument port (6) and is extended out of the distal end (26) of the shaft. The fiberscope may be pushed through tumor tissue to provide visualization from the inside and in front of the tumor.

In one advantageous embodiment, the fiberscope may be inserted through one of the inner lumens of the steerable catheter or the balloon catheter described above. Preferably, a length of the fiberscope is sufficiently longer than the length of both the working channel (1) and the catheter disposed in the working channel such that the fiberscope extends past the distal end of the catheter. The distal end of the catheter may include a lens cleaning device for cleaning the fiberscope lens. The cleaning device is made with any suitable type of material, for example, a textile bundle, that is affixed to the distal end of the catheter. The fiberscope is cleaned by moving it back and forth through the cleaning device, thus wiping the lens of the fiberscope.

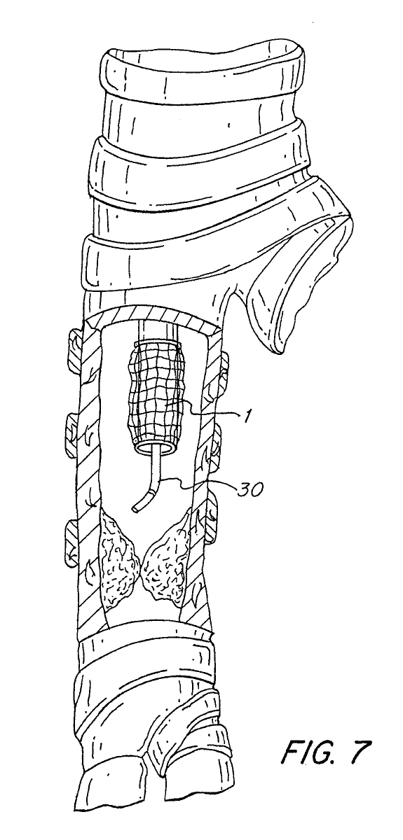

FIGS. 7-9 illustrate a method of insertion and operation of the working channel (1) in a bodily cavity in accordance with the present invention.

As shown in FIG. 7, the working channel (1) is introduced into a desired location within a patient's body. In order to assist the surgeon in insertion and positioning of the working channel (1), the imaging device (30) is inserted into one of the working channel's lumens and is extended out of the distal end of the working channel for visualizing the tissue adjacent the distal end of the working channel. As described above, the distal end of the imaging device may be manipulated by the surgeon to steer the imaging device (30) through the bodily passages to the target site. Additionally, the elongated shaft (2) of the working channel may have imaging markers to assist the surgeon in visualizing the exact position of the working channel within the bodily cavity.

It should be noted that a guide wire may be first inserted into the bodily cavity and anchored at the target site. Then, the working channel (1) is advanced over the guide wire and anchored at the target site, and the guide wire is removed from the bodily cavity.

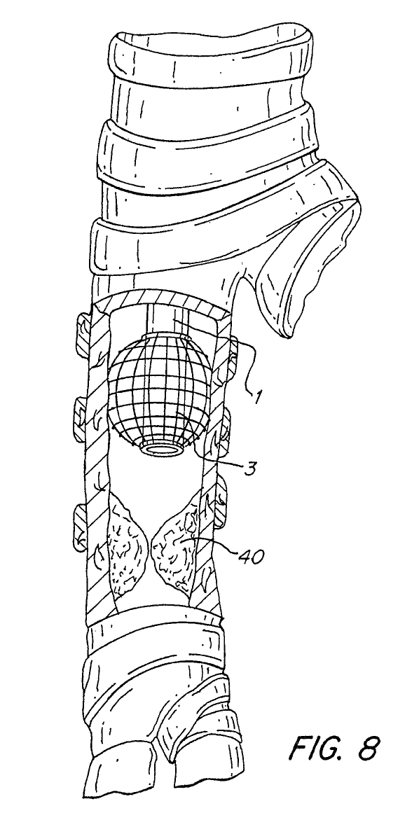

Once the working channel (1) is positioned at the target site (40), the balloon (3) provided at the distal end of the elongated shaft (2) is inflated by supplying fluid thereto from the pump or any other fluid source via the inflation port, as shown in FIG. 8. The balloon (3) is inflated until the outer wall of the balloon contacts the surrounding tissue such that the textured outer surface of the balloon (3) grips the tissue, thereby anchoring the working channel at the target site.

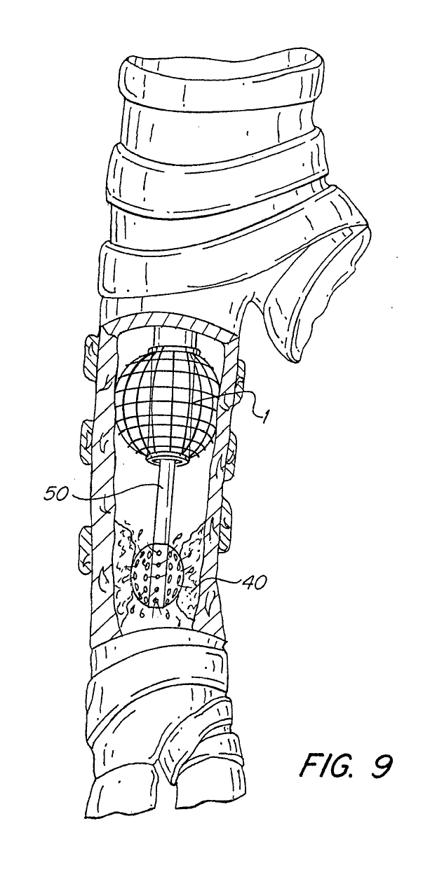

Next, the imaging device (30) is removed from the working channel lumen and a desired medical instrument or device is inserted therein for performing a medical procedure. For example, as shown in FIG. 9, a resector balloon system (50) described in U.S. Pat. No. 8,226,601 may be inserted through the working channel lumen of the working channel (1) to resect the tumor tissue (40). In some embodiments, the imaging device (30) is not removed from the working channel (1) and is used to visualize the surgical site during the procedure. Furthermore, as discussed above, a fiberscope may be first pushed through the tumor tissue to provide an image of the inside and in front of the tumor (40) prior to the resecting procedure to allow the surgeon to more precisely gauge the size, location and morphology of the tumor. Additional instruments and/or devices may also be introduced into the bodily cavity through the working channel (1) during the procedure to perform various functions, such as, for example, delivering therapeutic/diagnostic agents, providing irrigation fluid/suction, taking tissue samples, etc.

Once the procedure is completed, the instruments and/or devices are removed out of the working channel (1). Then, the balloon (3) is deflated and the working channel (1) is removed from the patient's body.

It would be appreciated by those skilled in the art that various changes and modifications can be made to the illustrated embodiment without departing from the spirit of the present invention. All such modifications and changes are intended to be covered hereby.

What is claimed is:

1. An anchored working channel comprising:
an elongated shaft with a proximal end and a distal end; and
at least one inflatable balloon positioned at the distal end of the elongated shaft and having an outer wall, said outer wall comprising an outer surface for contacting surrounding tissue;
wherein the elongated shaft has a first lumen through which fluid is supplied to inflate said at least one inflatable balloon such that said at least one balloon anchors the shaft to surrounding tissue;
wherein the elongated shaft has a second lumen that accommodates at least one medical instrument and/or device inserted therein; and
wherein said outer surface of said at least one inflatable balloon comprises a textured surface for preventing slippage of the outer surface on surrounding tissue.

2. The anchored working channel of claim 1, wherein said textured surface of said at least one inflatable balloon comprises a mesh disposed on the outer wall of said balloon.

3. The anchored working channel of claim 2, wherein the mesh is a weft knit mesh.

4. The anchored working channel of claim 2, wherein the mesh comprises polyethylene.

5. The anchored working channel of claim 2, wherein the mesh comprises elastane.

6. The anchored working channel of claim 1, further comprising an imaging device disposed in one of the first lumen and the second lumen.

7. The anchored working channel of claim 6, wherein a distal end of said imaging device extends out from the distal end of said elongated shaft for viewing tissue in front of the anchored working channel.

8. The anchored working channel of claim 6, wherein said imaging device comprises a fiber optic bundle.

9. The anchored working channel of claim 6, wherein said imaging device comprises a steerable distal section.

10. The anchored working channel of claim 9, further comprising a control unit for actuation of the steerable distal section of said imaging device by a user.

11. The anchored working channel of claim 9, wherein said imaging device comprises an inner lumen and a plurality of steering lumens.

12. The anchored working channel of claim 11, wherein said imaging device further comprises at least one pull wire disposed in at least one of the plurality of steering lumens for actuation of the distal section of said imaging device.

13. The anchored working channel of claim 1, wherein the fluid is a gas.

14. The anchored working channel of claim 1, wherein fluid is supplied to said at least one balloon by a pump.

15. The anchored working channel of claim 14, wherein said pump is an electro-pneumatic pump.

16. The anchored working channel of claim 14, wherein said pump further comprises a vacuum source that evacuates the fluid from said at least one inflatable balloon.

17. The anchored working channel of claim 14, wherein said pump includes at least one sensor for measuring at least one parameter and a processor that controls the supply of the fluid to said at least one inflatable balloon based on the at least one measured parameter.

18. The anchored working channel of claim 14, further comprising a data device from which said pump identifies a particular type of the working channel connected thereto.

19. The anchored working channel of claim 1, wherein said at least one inflatable balloon comprises at least one imaging marker.

20. The anchored working channel of claim 19, wherein said at least one imaging marker comprises a radio-opaque ring.

21. The anchored working channel of claim 1, wherein the proximal end of said elongated shaft comprises a first port in communication with the first lumen and at least one second port in communication with the second lumen.

22. The anchored working channel of claim 1, wherein said elongated shaft further comprises a bypass lumen in fluid communication with an opening in the elongated shaft positioned proximally from said inflatable balloon for passing bodily fluids therethrough.

23. The anchored working channel of claim 1, wherein said at least one inflatable balloon comprises a plurality of inflatable balloons positioned at different locations along said elongated shaft.

24. The anchored working channel of claim 23, wherein each of the plurality of inflatable balloons is inflatable separately from the other balloons.

25. The anchored working channel of claim 1, wherein said medical instrument and/or device comprises a resecting balloon catheter.

26. The anchored working channel of claim 1, wherein said medical instrument and/or device comprises a steerable catheter.

27. The anchored working channel of claim 1, wherein said medical instrument and/or device comprises a fiberscope.

28. The anchored working channel of claim 1, further comprising at least one opening in the outer wall of the elongated shaft for delivering a therapeutic and/or diagnostic agent to surrounding tissue.

29. A method of performing a medical procedure via an anchored working channel, comprising the steps of:
inserting a working channel into a bodily cavity, wherein said working channel comprises an elongated shaft having at least a first lumen and a second lumen therein, and an inflatable balloon positioned at a distal end of the elongated shaft and having an outer wall with a textured surface for preventing slippage of the outer wall on surrounding tissue;
advancing said working channel through the bodily cavity until the inflatable balloon reaches an anchoring position;
supplying fluid to the first lumen with a pump until the balloon is inflated such that the textured surface exerts sufficient pressure on the wall of the bodily cavity to retain the balloon in the anchoring position;
inserting at least one medical instrument and/or device through the second lumen and out of the distal end of said elongated shaft for performing the medical procedure;
withdrawing the at least one medical instrument and/or device from the second lumen;
deflating the inflatable balloon; and
withdrawing the working channel from the bodily cavity.

30. The method of claim 29, wherein the pump includes at least one sensor for measuring at least one parameter and a processor for controlling the supply of fluid to the inflatable balloon based on at the least one measured parameter.

31. The method of claim 30, wherein the at least one sensor measures at least one patient's physiologic parameter.

32. The method of claim 29, further comprising the step of using an imaging device disposed in one of the first lumen and the second lumen to visualize tissue in the bodily cavity.

33. The method of claim 32, wherein the step of using the imaging device comprises extending a distal tip of said imaging device out of the distal end of said elongated shaft to visualize tissue in front of said anchored working channel.

34. The method of claim 32, wherein said imaging device comprises a steerable distal section and the step of using the imaging device comprises actuating said distal section via a control unit to maneuver said imaging device in the bodily cavity.

35. The method of claim 34, further comprising the step of using at least one imaging marker to position the inflatable balloon within the bodily cavity.

36. The method of claim 29, wherein said elongated shaft comprises a bypass lumen in fluid communication with an opening in the elongated shaft positioned proximally from said inflatable balloon, further comprising the step of passing bodily fluids through the bypass lumen and out of the opening in said elongated shaft.

37. The method of claim 36, further comprising the step of measuring airflow through the bypass lumen.

38. The method of claim 29, wherein said textured surface of said inflatable balloon comprises a mesh disposed on the outer wall of said balloon.

39. The method of claim 38, wherein the mesh is a weft knit mesh.

40. The method of claim 38, wherein the mesh comprises elastane.

41. The method of claim 29, wherein the step of advancing said working channel through the bodily cavity comprises the steps of inserting a guide wire into the bodily cavity and advancing said working channel over the guide wire until it reaches the anchoring position.

42. The method of claim 29, further comprising the step of delivering a therapeutic and/or diagnostic agent to tissue via at least one opening in the outer wall of the elongated shaft.

43. The method of claim 42, wherein the step of delivering the therapeutic and/or diagnostic agent to tissue comprises at least partially deflating the inflatable balloon and moving the elongated shaft in a proximal direction to facilitate extravasation of the agent into tissue.
